(19) United States
(12) Patent Application Publication
(10) Pub. No.: US 2016/0287447 A1
(43) Pub. Date: Oct. 6, 2016

(54) MEDICATED PAD FOR A CHINSTRAP

(71) Applicant: Timothy MOORADD, New York, NY (US)

(72) Inventor: Timothy MOORADD, New York, NY (US)

(21) Appl. No.: 14/676,184

(22) Filed: Apr. 1, 2015

(51) Int. Cl. A61F 13/02; A61K 31/235; A61F 13/12; A61L 15/44; A42B 3/08; A42B 3/04; A61K 31/60; A61K 31/7048

(52) U.S. Cl. CPC A61F 13/025; A61K 31/60; A61K 31/235; A61K 31/7048; A61L 15/44; A42B 3/08; A42B 3/0406; A61F 13/122; A61L 2300/21; A61L 2300/40; A61F 2013/00272

(57) ABSTRACT

A medicated pad for use in a chinstrap holder is provided. The medicated pad includes a non-abrasive user side having a medication. A self-adhering side is arranged opposite the user side. A plurality of reliefs in the medicated pad enables the medicated pad to be shaped to complement the chinstrap holder and be adhered to the chinstrap holder.

### FIELD OF INVENTION

This application generally relates to a pad for use in athletic equipment and is more particularly related to a medicated pad for use in a chinstrap associated with a protective helmet.

### BACKGROUND

Acne is a skin condition that causes breakouts, skin irritations, pimples, blemishes, and/or greasy skin. Acne mechanica is a specific type of acne caused by heat, friction, rubbing, and constant pressure of skin against material. Acne mechanica is particularly a problem for adolescents involved in sports. Organized sports, such as football, hockey, lacrosse, and field hockey, require participants to wear protective headgear that includes a chinstrap. Other activities, such as motorcycle or bicycle riding, skate boarding, and motor cross, also require prolonged helmet usage. The helmet's chinstrap constantly rubs and applies pressure to a user's chin. This causes the user's chin to experience severe irritation, resulting in breakouts and blemishes. Known solutions for addressing this problem include providing a semi-ventilated holder for a user's chin. Such solutions do not provide a fully encapsulating chin pad, resulting in inadequate padding and protection of a user's chin. Another solution for alleviating acne mechanica includes treating the user's skin before and after prolonged pressure and irritation of the user's skin. One such treatment includes wiping a user's skin with medicated pads, such as OXY® daily defense pads. These known pads include a relatively highly concentrated solution of acne medication which is intended for limited contact with a user's skin.

A chinstrap insert that can have prolonged contact with a user's chin that alleviates the conditions causing acne mechanica and dermatological or bacterial infections, and provides full protection is desired.

### SUMMARY

A medicated pad configured for use in a chinstrap is provided. The medicated pad has a non-abrasive user side that includes the medication and a self-adhering side that is opposite the user side. The medication is provided in a lower concentration than known acne medication wipes, in order to prevent further aggravating a user's skin due to prolonged contact with a highly concentrated medication. The medicated pad includes a plurality of reliefs that enable it to complement the shape of a chin support in the chinstrap and so it can be adhered to the chin support which generally is concave.

### BRIEF DESCRIPTION OF THE DRAWINGS

The following detailed description of the invention will be best understood when read in conjunction with the appended drawings. In the drawings:

FIG. 1 shows a perspective view of a medicated pad according to the invention in a pre-installed configuration.

FIG. 2 illustrates a perspective view of the medicated pad of FIG. 1 as it may appear in an installed configuration.

FIG. 3 is a side elevation of the medicated pad of FIG. 1

FIG. 4 is a top plan view of the medicated pad of FIG. 1.

FIG. 5 illustrates the medicated pad of FIG. 1 installed in a support of a chinstrap.

FIG. 6 illustrates perspective view of the medicated pad in use.

### DETAILED DESCRIPTION OF THE PREFERRED EMBODIMENTS

With reference to FIGS. 1, 2 and 3, the pad 1 is generally rectangular with a non-abrasive user side 2 that includes a medication 4 and a opposite side 6 that includes an adhesive. If desired, the medicated pad 1 can have a predetermined, specific shape that substantially conforms to a given chinstrap. In one preferred embodiment, the non-abrasive user side 2 has a foam backing 18, such as cross-linked polyethylene foam. The thickness of the foam backing 18 can vary according to a particular desire to provide the desired cushioning for a given activity. In one preferred embodiment, the self-adhering side 6 has an acrylic adhesive 20.

In another embodiment, the non-abrasive user side 2 is a wicking surface for directing perspiration away from a user's chin. In this embodiment, the non-abrasive user side 2 is hydrophilic and attracts perspiration away from a user's chin.

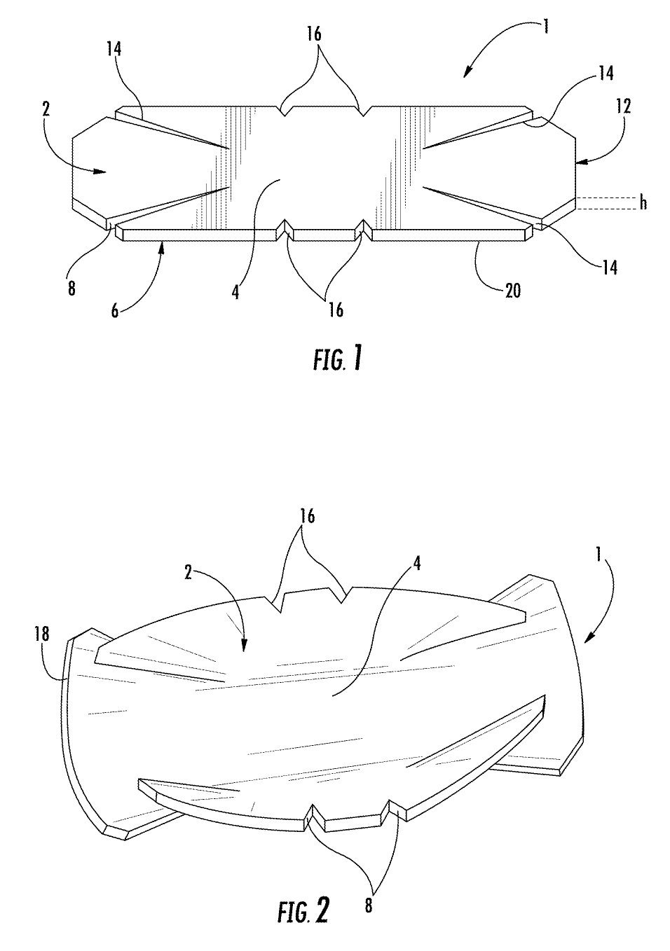

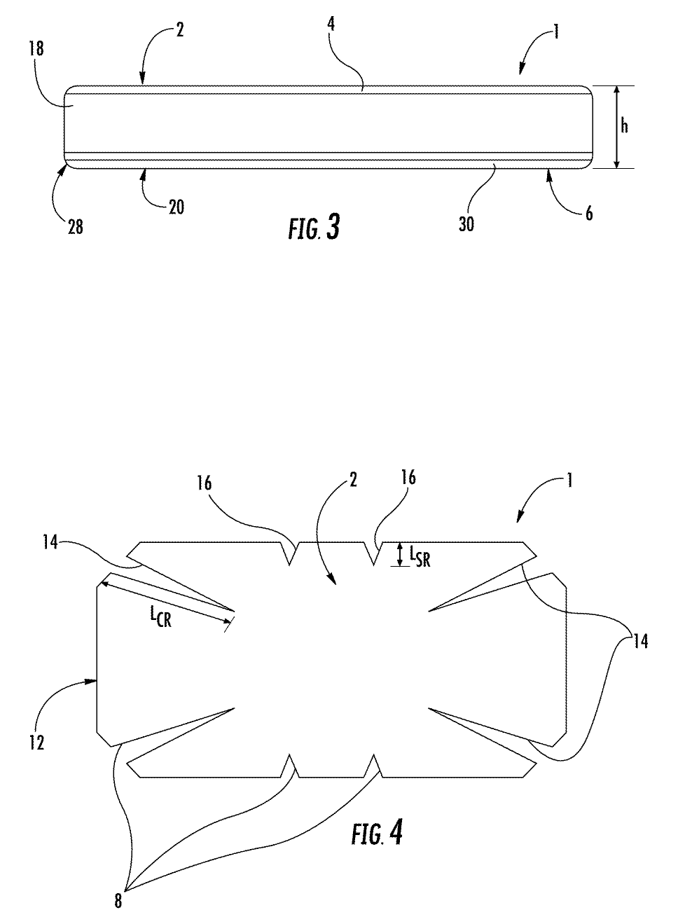

The self-adhering side 6 is opposite the user side 2. A plurality of reliefs or notches 8 in the medicated pad 1 enable the medicated pad to be shaped to complement the chin support holder 10 and for the self-adhering side 6 to adhere to the holder 10. The plurality of reliefs 8 are positioned around the medicated pad's periphery 12 preferably have a generally triangular shape. The plurality of reliefs 8 extend inwardly from the medicated pad's periphery 12. The plurality of reliefs 8 preferably includes corner reliefs 14 and longitudinal side reliefs 16. In a preferred embodiment, there are four corner reliefs 14 and four side reliefs 16 provided. The number and configuration of reliefs can be varied to accommodate a predetermined, specific chinstrap. The corner reliefs 14 are preferably deeper inwardly than the longitudinal side reliefs 16. As shown in FIG. 4, the side reliefs 16 preferably have a length (L_{SR}) that is about 20% of the length (L_{CR}) of the corner reliefs 14. As shown in FIGS. 2, 5, and 6, the reliefs 8 enable the medicated pad 1 to conform to a concave, cup-like shape.

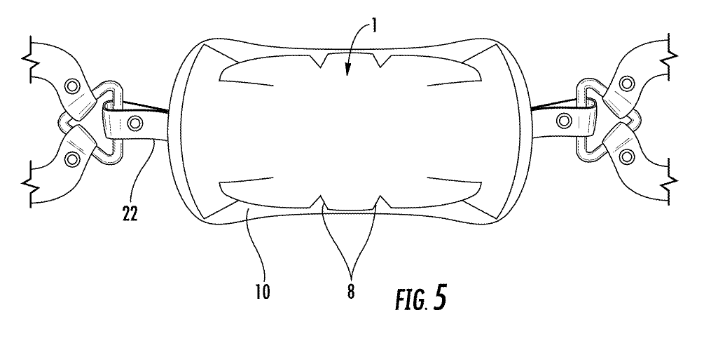

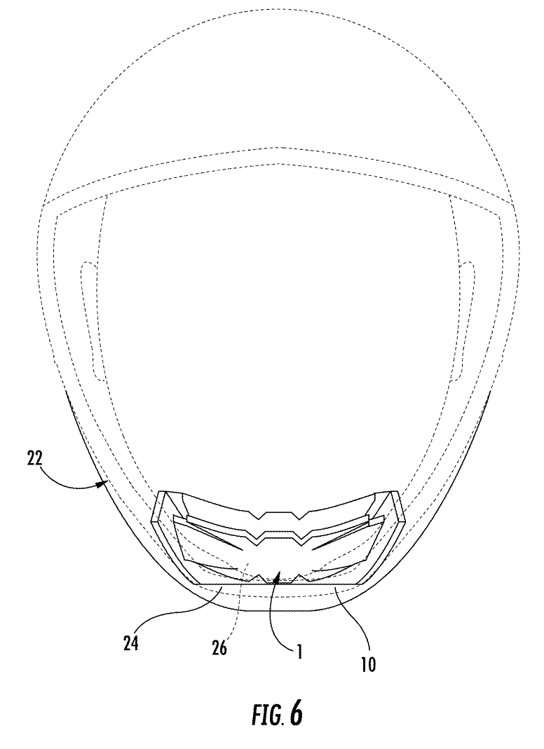

As shown in FIGS. 5 and 6, the chinstrap 22 includes a concave portion 24 configured to receive and support a user's chin 26. The chinstrap 22, concave portion 24, and chin 26 are shown in phantom lines in FIG. 6. The medicated pad 1 conforms to complement the chinstrap's concave portion 24. The self-adhering side 6 is removably held to the chinstrap's concave portion 24, so it can be removed and replaced as desired. The self-adhering side 6 includes a release strip 28 that is away from the medicated pad 1 to expose the self-adhering side 6 to the chinstrap's concave portion 24. The release strip 28 is of a type commonly known type, such as a clay coated bleached kraft with a silicone release coating 30 on one side. The layers of the medicated pad 1 are illustrated in FIG. 3. In one preferred embodiment, the medicated pad 1 is 0.060-0.090 inches thick.

In a preferred embodiment, the medication 4 has a concentration of 0.5-2.0% of salicylic acid or benzoyl peroxide, or a combination of them. In another preferred embodiment, the medication 4 has a concentration of 0.5-2.0% of erythromycin. These concentrations are lower than the concentration typically found in acne medicated wipes in order to prevent irritation of a user's skin caused by the medication. In another preferred embodiment, the medication 4 has a coating that dissolves or is activated based on quantity of the user's perspiration.

The present embodiments are therefore to be considered in all respects as illustrative and not restrictive, the scope of the invention being indicated by the appended claims rather than by the foregoing description, and all changes which come within the meaning and range of equivalency of the claims are therefore to be embraced therein.

What is claimed is:

1. A medicated pad comprising:
a non-abrasive user side that includes a medication;
a self-adhering side opposite the user side; and
a plurality of reliefs that enable the medicated pad to complement the shape of a holder with the self-adhering side adhered to the holder.

2. The medicated pad of claim 1, wherein the plurality of reliefs are positioned around the medicated pad's periphery.

3. The medicated pad of claim 1, wherein the plurality of reliefs extend inwardly from the medicated pad's periphery.

4. The medicated pad of claim 1, wherein the medicated pad has a rectangular shape.

5. The medicated pad of claim 1, wherein the medicated pad is generally rectangular and the plurality of reliefs includes corner reliefs and longitudinal side reliefs.

6. The medicated pad of claim 5, wherein there are four corner reliefs and four longitudinal side reliefs.

7. The medicated pad of claim 5, wherein the corner reliefs extend further inwardly than the longitudinal side reliefs.

8. The medicated pad of claim 5, wherein the longitudinal side reliefs have a length that is 20% of a length of the corner reliefs.

9. The medicated pad of claim 1, wherein the medication includes a concentration of 0.5-2.0% of salicylic acid or benzoyl peroxide, or a combination thereof.

10. The medicated pad of claim 1, wherein the medication includes a concentration of 0.5-2.0% of erythromycin.

11. The medicated pad of claim 1, wherein the non-abrasive user side has a foam backing.

12. The medicated pad of claim 1, wherein the self-adhering side includes an acrylic adhesive.

13. The medicated pad of claim 1, wherein the medicated pad is configured to adapt to a concave curvature of the holder.

14. The medicated pad of claim 1, wherein the medicated pad is 0.060-0.090 inches thick.

15. A combination of a chinstrap and medicated pad comprising:
a chinstrap that includes a concave portion configured to receive a user's chin; and,
a medicated pad comprising a non-abrasive user side that includes a medication, a self-adhering side opposite the user side, and a plurality of reliefs that enable the medicated pad to complement the chinstrap's concave portion.

16. The combination of the chinstrap and medicated pad of claim 15, wherein the self-adhering side is detachably fixed to the chinstrap's concave portion.

17. The combination of the chinstrap and medicated pad of claim 15, wherein the self-adhering side includes a release strip, wherein pulling the release strip away from the medicated pad releases the self-adhering side from the chinstrap's concave portion.

18. The combination of the chinstrap and medicated pad of claim 17, wherein the release strip includes a clay coated bleached kraft with a silicone release coating on one side.

19. A pad comprising:
a non-abrasive user side;
a self-adhering side opposite the user side; and
a plurality of reliefs that enable the self-adhering side of the pad to adhere to the holder.

20. The pad of claim 19, wherein the non-abrasive user side includes a wicking surface.
